(19) United States
(12) Patent Application Publication
(10) Pub. No.: US 2005/0166863 A1
(43) Pub. Date: Aug. 4, 2005

(54) Sterilizing comb for pets

(75) Inventors: Hong Ill Park, Buchun (KR); Kyu-Young Ko, Buchun (KR)

Correspondence Address: PARK & SUTTON LLP, 3255 WILSHIRE BLVD, SUITE 1110, LOS ANGELES, CA, 90010, US

(73) Assignee: WON SHIK SHIN

(21) Appl. No.: 10/918,090

(22) Filed: Aug. 13, 2004

(30) Foreign Application Priority Data
Jan. 20, 2004 (KR) 10-2004-0004470

(51) Int. Cl. A01K 13/00

(52) U.S. Cl. 119/625

(57) ABSTRACT

A sterilizing comb for pets includes a case, a plurality of bristles attached on the case and a sterilizer. The sterilizer is a lamp that emits high-energy rays. The high-energy ray may be the ultraviolet ray or the infrared ray. The case includes a body and a handle. The body includes a brush plate and one or more light emitting openings. The bristles are attached on the brush plate. The high-energy rays are emitted through the light emitting openings. The sterilizing comb further includes an air suction fan and a photo catalyst filter. The air suction fan sucks air through the light emitting openings. The photo catalyst filter generates photo oxidation with the high-energy rays. The air suction fan is positioned adjacent the photo catalyst filter so that the sucked air passes through the photo catalyst filter.

### BACKGROUND OF THE INVENTION

The present invention relates to a sterilizing comb for pets. More particularly, this invention relates to a sterilizing comb for pets, which can sterilize the hair and skin of a pet with ultraviolet ray or infrared ray, and collect debris and eliminate it.

Recently, pets are recognized as members of the family, and families keeping pets are increasing. The money expended for pets and the market for pet products are rapidly expanding per these trends.

Pet products, which are required for taking care of pets, include food, clothing, decoration accessories, health product, and cosmetic products etc. Among the pet products, needs for products and services related to the pet health have been substantially increased since the pets are now recognized as members of the family.

Especially, maintenance for pet hair is important because improperly maintained hair become dirty and messy and may provide environment in which parasites or bacteria can inhibit. Pets may get skin disease or become ill due to such parasites or bacteria, and expensive medical treatment may be required as a result of pure hair management.

### SUMMARY OF THE INVENTION

The present invention contrives to solve the disadvantages of the prior art.

An objective of the invention is to provide a sterilizing comb for pets that emit high-energy rays including ultraviolet ray or infrared ray so that the hair and skin of the pets are effectively sterilized.

Another objective of the invention is to provide a sterilizing comb that can collect fallen hair or other debris and eliminate or sterilize them.

In order to achieve the above objectives, the present invention provides a sterilizing comb for pets that includes a case, a plurality of bristles attached on the case and a sterilizer.

The sterilizer is a lamp that emits high-energy rays. The high-energy ray includes the ultraviolet ray or the infrared ray.

Alternatively, the sterilizer is an ozone-generator that ejects ozone.

The case includes a body and a handle. The body includes a brush plate and one or more light emitting openings. The bristles are attached on the brush plate. The high-energy rays are emitted through the light emitting openings.

The sterilizing comb further includes an air suction fan and a photo catalyst filter.

The air suction fan sucks air through the light emitting openings. The photo catalyst filter generates photo oxidation with the high-energy rays. The air suction fan and the photo catalyst filter are installed inside the handle.

The sterilizing comb further includes a motor that rotates the air suction fan. The air suction fan is positioned adjacent the photo catalyst filter so that the sucked air passes through the photo catalyst filter.

The sterilizing comb further includes a power supply, and a switch that controls the power supply. The power supply supplies power to the lamp. The switch is installed on the handle. The motor and the power supply are installed inside the handle.

The advantages of the present invention are: (1) the sterilizing comb improves hygienic state of pets by sterilizing or eliminating various parasites or bacteria with ultraviolet rays, which are emitted from UV lamps; (2) the sterilizing comb protects the pet's hair and facilitate curing skin disease with infrared rays, which are emitted from IR lamps; and (3) the sterilizing comb enables hygienic maintenance of pets and provides clean interior environment by collecting fallen hair or dust that is generated during the process of caring hair, and removing such debris with photo catalytic reaction.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 is a perspective view showing a sterilizing comb for pets according to the present invention;

FIG. 2 is a plan view of the sterilizing comb; and

FIG. 3 is an elevation view of the sterilizing comb.

### DETAILED DESCRIPTION OF THE INVENTION

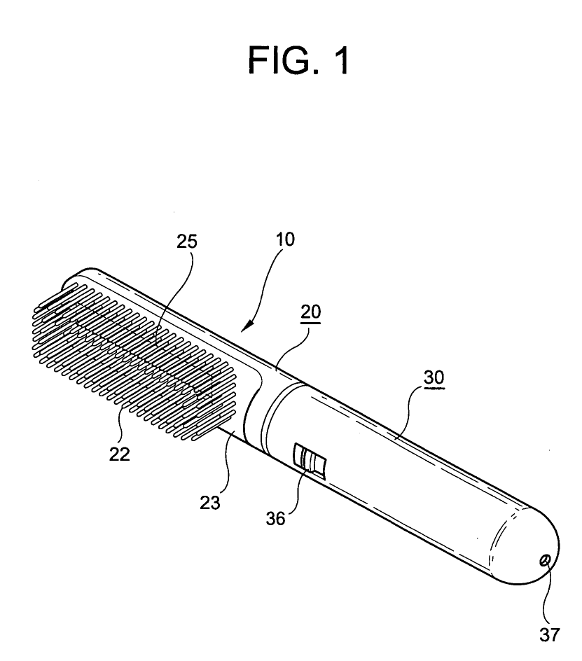

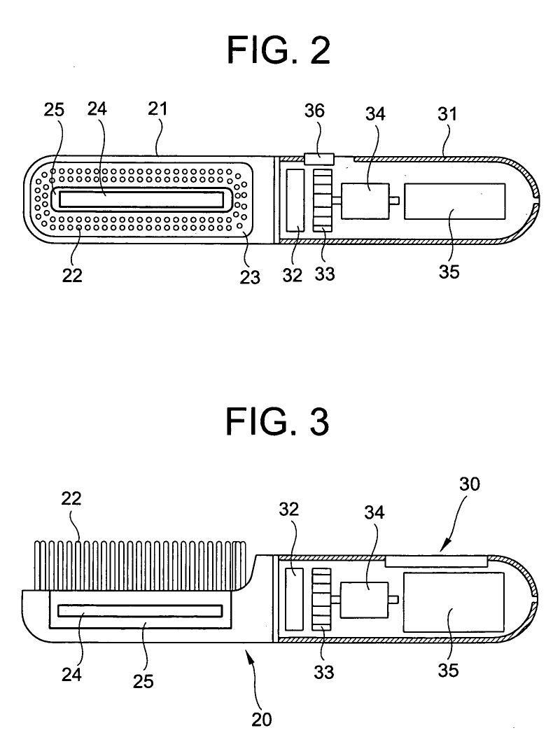

FIGS. 1-3 show a sterilizing comb 10 for pets according to the present invention. The sterilizing comb 10 includes a case 21, a plurality of bristles 22 attached on the case 21 and a sterilizer.

The sterilizer is a lamp 24 that emits high-energy rays. The high-energy ray includes the ultraviolet ray or the infrared ray.

Alternatively, the sterilizer is an ozone-generator that ejects ozone.

The case 21 includes a body 20 and a handle 30. The handle 30 is detachably attached to the body 20.

The body 20 includes a brush plate 23 and a light emitting opening 25. The light emitting opening 25 is formed on the brush plate 23. The light emitting opening 25 is elongated along the length of the brush plate 23. Alternatively, a plurality of light emitting openings 25 may be provided. The dimension of the light emitting openings 25 is determined to correspond to the length of the lamp 24. In order to increase the amount of the ultraviolet rays in the direction of the light emitting opening 25, reflecting surfaces may be provided around the lamp 24. Also, more than one lamp 24 may be installed.

The bristles 22 are attached on the outer surface of the brush plate 23. Plastic or metal may be used as the material of the bristles 22. The high-energy rays are generated by the lamp 24, which is installed inside the sterilizing comb 10, are emitted to outside of the sterilizing comb 10 through the light emitting opening 25.

Photo catalyst is applied on the bristles 22 so that stains or foreign objects attached on the bristles 22 may be oxidized with photo oxidation reaction with the ultraviolet ray emitted from the lamp 24.

The sterilizing comb 10 further includes an air suction fan 33 and a photo catalyst filter 32.

The air suction fan 33 sucks air through the light emitting opening 25. The photo catalyst filter 32 generates photo oxidation with the high-energy rays emitted by the UV lamp 24. The air suction fan 33 and the photo catalyst filter 32 are installed inside the handle 30.

The photo catalyst filter 32 is a filter on which photo catalyst is applied or sprayed. The photo catalyst used for the photo catalyst filter 32 includes zinc oxide (ZnO), tungsten oxide (WO₃), cadmium oxide (CdO), indium oxide (In₂O₃), silver oxide (Ag₂O) manganese oxide (MnO₂), copper oxide (Cu₂O), iron oxide (Fe₂O₃), tin oxide (SnO₂), vanadium oxide (V₂O₅), niobium oxide (Nb₂O₃) etc. Preferably, titanium dioxide (TiO₂) is used for chemically stable and excellent blocking operation.

Metal fibers applied or coated with photo catalyst, which is described on the applicant's earlier Korean application, may be filled to increase contact surface area thereby maximizing the effect of photo catalytic reaction.

The sterilizing comb 10 further includes a motor 34 that rotates the air suction fan 33. The air suction fan 33 is positioned adjacent the photo catalyst filter 32 so that the sucked air passes through the photo catalyst filter 32.

The sterilizing comb 10 further includes a power supply 35, and a switch 36 that controls the power supply 35. The power supply 35 supplies power to the lamp 24. The switch 36 is installed on the handle 30. The motor 34 and the power supply 35 are installed inside the handle 30. The handle 30 includes a handle case 31 that encloses the photo catalyst filter 32, the air suction fan 33, the motor 34, the power supply 35, and the switch 36.

An air outlet 37 is provided on the rear portion, which is opposite to the portion engaged with the body 20, of the handle case 31. Air sucked by the air suction fan 33 is ejected through the air outlet 37.

The lamp 24 and the air suction fan 33 are operated together or separately depending on the setting by the switch 36. The power supply 35 provides power to both the lamp 24 and the air suction fan 33. Ordinary dry batteries or rechargeable batteries may be used for the power supply 35.

When the lamp 24 and the air suction fan 33 are operated by turning on the switch 36, and the sterilizing comb 10 is used for combing a pet's hair, parasites or bacteria that inhibit on the hair or the skin of the pet are sterilized with the ultraviolet rays emitted by the lamp 24. In addition, foreign objects or dust that are adhered to the hair are sucked into the sterilizing comb 10 via the light emitting opening 25 by the air suction fan 33, and removed with photo catalytic reaction when they reach the photo catalyst filter 32.

While the invention has been shown and described with reference to different embodiments thereof, it will be appreciated by those skilled in the art that variations in form, detail, compositions and operation may be made without departing from the spirit and scope of the invention as defined by the accompanying claims.

1. A sterilizing comb for pets comprising:
a) a case;
b) a plurality of bristles attached on the case; and
c) a sterilizer.

2. The sterilizing comb of claim 1, wherein the sterilizer comprises a lamp that emits high-energy rays.

3. The sterilizing comb of claim 1, wherein the sterilizer comprises an ozone-generator that ejects ozone.

4. The sterilizing comb of claim 1, wherein the case comprises a body, and the body comprises a brush plate and one or more light emitting openings, wherein the bristles are attached on the brush plate, wherein the sterilizer comprises a lamp that emit high-energy rays, wherein the high-energy rays are emitted through the light emitting openings.

5. The sterilizing comb of claim 4, wherein the high-energy ray is the ultraviolet ray.

6. The sterilizing comb of claim 4, wherein the high-energy ray is the infrared ray.

7. The sterilizing comb of claim 1, further comprising an air suction fan.

8. The sterilizing comb of claim 7, wherein the case comprises a body, and the body comprises a brush plate and one or more light emitting openings, wherein the bristles are attached on the brush plate, wherein the sterilizer comprises a lamp that emit high-energy rays, wherein the high-energy rays are emitted through the light emitting openings, wherein the air suction fan sucks air through the light emitting openings.

9. The sterilizing comb of claim 8, further comprising a photo catalyst filter, wherein the photo catalyst filter generates photo oxidation with the high-energy rays.

10. The sterilizing comb of claim 9, wherein the case further comprises a handle, wherein the air suction fan and the photo catalyst filter are installed inside the handle.

11. The sterilizing comb of claim 10, further comprising a motor that rotates the air suction fan, wherein the air suction fan is positioned adjacent the photo catalyst filter so that the sucked air passes through the photo catalyst filter.

12. The sterilizing comb of claim 11, further comprising a power supply, and a switch that controls the power supply, wherein the power supply supplies power to the lamp.

13. The sterilizing comb of claim 12, wherein the switch is installed on the handle, wherein the motor and the power supply are installed inside the handle.

14. The sterilizing comb of claim 1, wherein photo catalyst is applied on the bristles.
